(19) United States
(12) Patent Application Publication
(10) Pub. No.: US 2007/0154523 A1
(43) Pub. Date: Jul. 5, 2007

(54) Controlled pupil dilation for diagnostic and treatment of visual anomalies

(76) Inventor: Rick Lewis, Sacramento, CA (US)

Correspondence Address: JOHN P. O'BANION;O'BANION & RITCHEY LLP, 400 CAPITOL MALL SUITE 1550, SACRAMENTO, CA, 95814, US

(21) Appl. No.: 11/324,128

(22) Filed: Dec. 30, 2005

(51) Int. Cl. A61K 31/137; A61F 2/02

(52) U.S. Cl. 424/427; 514/649

(57) ABSTRACT

Methods and compositions are disclosed for topically applying an ophthalmic solution to the eye to at least temporarily reduce the effects of various visual anomalies. The solution contains a therapeutically effective amount of one or more pupil dilating agents that generate a moderate amount of dilation in order to relieve symptoms such as vitreous floaters. Moreover, the composition may be topically applied in approximately a one drop dose, which may be instilled on an as needed basis depending on the concentration and rate of release of one or more of the following or similar dilating agents: naphazoline, ephedrine, naphazoline HCl, phenylephrine HCl, epinephrine and/or dipivefrin.

### CROSS-REFERENCE TO RELATED APPLICATIONS

Not Applicable

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND OF THE INVENTION

1. Field of the Invention

This invention pertains generally to treatment of visual anomalies, and more particularly to temporary treatment of visual anomalies via controlled pupillary dilation.

2. Description of Related Art

One of the most common complaints presented by patients visiting an ophthalmologist is the existence of vitreous floaters. Vitreous floaters are common visual anomalies caused by tiny clumps of fibers or cells inside the vitreous, the clear gel-like fluid that fills the inside of the eye. To the patient, vitreous floaters typically appear as small dots, circles, lines, clouds or cobwebs. They may also appear as small specks or clouds moving in the field of vision. While they look as if they are in front of the eye, they are actually floating inside of it. The patient is actually observing the shadows that the floaters cast on the retina, the layer of cells lining the back of the eye that senses light.

The most common cause of floaters is aging of the vitreous. When people reach middle age, the vitreous gel may start to condense or shrink; forming clumps or strands from the supportive fibers of the vitreous. The vitreous gel pulls away from the back wall of the eye, causing a posterior vitreous detachment. While this detachment is more common in older people, people who are nearsighted and those who have inflammation inside the eye can experience floaters as well.

These anomalies, while tolerable by some, can be very distracting to some patients, particularly those who have a need for clear vision during particular sight-intensive activities.

The methods of treating vitreous floaters currently involve a vitrectomy, a surgical procedure that involves removing the vitreous, blood and/or membranes from the eye; or, a laser procedure where the floaters are targeted by a high intensity laser light in order to obliterate them. Because of the invasive nature of these procedures, and the risk of complications associated with them, neither of these treatments are widely used.

Low vision is general term used to describe lowered visual acuity. Low vision is designated as someone with vision of 20/70 or less in the better eye with correction. People with low vision have reduced or marginal vision from a variety of causes, probably the most common cause if age related macular degeneration (ARMD) which is a frequent source of visual loss in the elderly. Some people who fall into this category often use their residual vision—their remaining sight—to complete daily tasks. Thus, some low vision patients who also have small pupils may benefit from increased pupil dilation.

### BRIEF SUMMARY OF THE INVENTION

The present invention relates to methods and compositions for providing relief from common visual anomalies, such as low vision and vitreous floaters. Specifically, the methods and compositions of the present invention provide for delivery of a dilating agent, e.g. through topical administration of a dilating drop, affecting a moderate degree of pupil dilation to obtain temporary relief from floaters for a safer, less invasive and less severe treatment than other options available to treat floaters such as laser or vitreous surgery.

The present invention provides compositions and methods for using an ophthalmic solution comprising a dilating agent such as an antihistamine, vasoconstrictor, glaucoma agent and/or diagnostic agent to control dilation of patient's pupils to a desired diameter, in order to provide relief from the undesirable visual anomalies such as vitreous floaters from simple application of the agent to the eyes on an as-needed basis.

Accordingly, the methods of the invention comprise topically applying an ophthalmic solution to the eye, wherein the solution contains a therapeutically effective amount of one or more pupil dilating agents, in order to achieve a moderate amount of dilation to relieve the symptoms of vitreous floaters. Moreover, the composition may be topically applied in approximately a one drop dose, which may be instilled on an as needed basis depending on the concentration and rate of release of one or more of the following or similar dilating agents: naphazoline, ephedrine, naphazoline HCl, phenylephrine HCl, epinephrine and/or dipivefrin.

The invention further provides for compositions comprising one or more dilating agents in a physiologically acceptable carrier, which can be administered to a subject at an effective dose to be therapeutically effective in concentration and rate of release. The compositions used in the practice of the present invention comprise both a rapid and sustained release of the effective dose.

The present invention, by enabling the use of an ophthalmic solution comprising one or more of the identified dilating agents, to increase pupillary dilation; provides a less invasive, less risky, safer treatment for vitreous floaters than the currently available surgical options.

An aspect of the invention is a method of temporarily treating a visual anomaly of a patient's eye. The method includes the steps of delivering a dilating agent to the patient's eye, controlling dilation of the patient's pupil to have a diameter within in a predetermined range to effect at least a temporary reduction in the presence of said visual anomaly.

The agent may be delivered topically directly to the eye, or orally administered. Topical administration may be through drops, spray, ointment, or like delivery method known in the art.

In a preferred embodiment, the method is used to treat vitreous floaters. Alternatively, the method may be used to treat patients with low vision. In addition, the method may be used for diagnostic testing that benefit from controlled dilation of the pupil.

In another preferred embodiment, the diameter of the pupil is controlled to be moderately dilated. Specifically, moderate dilation may comprise controlling the diameter of the pupil to be sub-mydriatic. In particular, the pupil may be controlled to have a diameter less than approximately 7 mm, and preferably in the range of approximately 3 mm to approximately 6.5 mm, and more preferably in the range of approximately 4 mm to approximately 6 mm.

By example and without limitation, the dilating agent may comprise of one or more of the following: antihistamine, vasoconstrictor, glaucoma agent, or diagnostic agent, e.g. naphazoline, ephedrine, naphazoline HCl, phenylephrine, epinephrine, propine or dipivefrin.

For topical administration, the dilating agent is preferably delivered in an ophthalmic carrier solution. In addition, the carrier solution is ideally configured such that the dilating agent is delivered in a one-drop dose.

In one embodiment, the dilating agent is delivered in a therapeutically effective dosage, wherein said therapeutically effective dosage comprises a minimum amount of dilating agent necessary to effect dilation of the patient's pupil to be within the predetermined range. The therapeutically effective dosage may also vary according to the patient's iris color, age, illness or concurrent medications.

Another aspect of the invention is a method of controlling dilation of a pupil of an eye. The method comprises topically delivering a dilating agent to the surface of the eye and controlling dilation of the pupil, wherein the dilating agent is delivered at a concentration that controls dilation of the pupil to a pre-determined diameter corresponding to a sub-mydriatic level.

In some embodiments, the pre-determined diameter is less than approximately 6 mm, and greater than approximately 4 mm.

In another embodiment, the dilating agent is delivered at a concentration that results in a pre-determined pupil diameter that inhibits the effect of vitreous floaters. Alternatively, the dilating agent is delivered at a concentration that results in a pre-determined pupil diameter suitable for diagnostic testing of the eye.

The dilating agent concentration may also be varied to accommodate different patient sensitivities that are associated with the patient's iris color, age, or concurrent medications taken by the patient.

Another aspect of the invention is an ophthalmic solution comprising an aqueous ophthalmic carrier solution configured to be topically delivered to a surface on a patient's eye, and a dilating agent configured to control dilation of a pupil of the patient. The dilating agent has a concentration that is configured dilate the pupil to a diameter in a pre-determined range, e.g. a sub-mydriatic range.

The carrier solution may be configured to deliver the dilating agent in a one-drop dose, having an exemplary pH of approximately 5.6-7.0.

In one embodiment, the dilating agent has a concentration that is configured dilate the pupil to a diameter that inhibits the effect of vitreous floaters. Alternatively, the dilating agent has a concentration that is configured dilate the pupil at different incremental diameters for diagnostic testing of the eye.

Yet another aspect is a method of prescribing a medication for treating visual anomalies of a patient's eye. The method comprises administering a baseline dosage of a dilating agent topically to the eye, measuring the dilation of a the patient's pupil, comparing the measured dilation against a desired dilation range, and changing the dosage until the measured dilation is within the desired range, wherein the desired dilation range is configured to reduce the effect of said visual anomalies.

In one embodiment, the baseline dosage is a function of one or more of the following patient conditions: patient iris color, patient age, patient ocular conditions, and concurrent patient medications.

Further aspects of the invention will be brought out in the following portions of the specification, wherein the detailed description is for the purpose of fully disclosing preferred embodiments of the invention without placing limitations thereon.

### BRIEF DESCRIPTION OF THE SEVERAL VIEWS OF THE DRAWING(S)

The invention will be more fully understood by reference to the following drawings which are for illustrative purposes only:

FIG. 1 is a schematic diagram of a human eye under relatively bright lighting conditions.

FIG. 2 is a schematic diagram of a human eye under relatively low lighting conditions.

FIG. 3 is a diagram illustrating a method of iteratively determining an effective dosage for treatment of ocular conditions in accordance with the present invention.

FIG. 4 is a diagram illustrating an alternative method of determining an effective dosage for treatment of ocular conditions in accordance with the present invention.

### DETAILED DESCRIPTION OF THE INVENTION

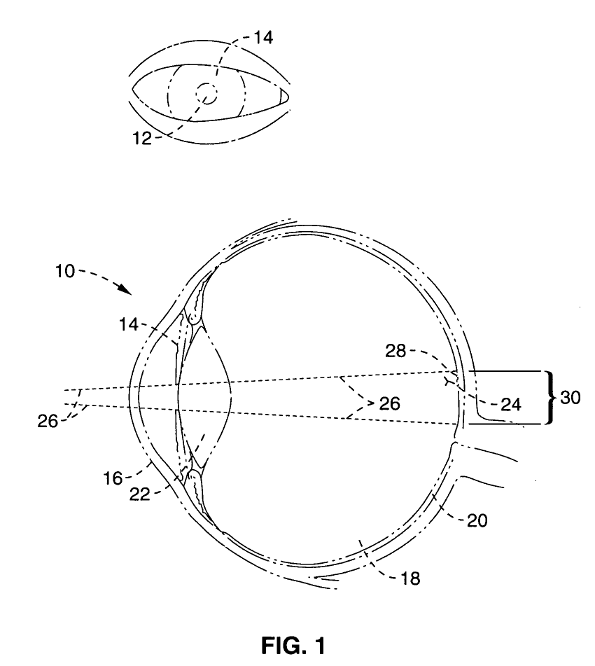

Referring more specifically to the drawings, for illustrative purposes the present invention is embodied in the compositions and methods generally shown in FIG. 1 through FIG. 4. It will be appreciated that the composition may vary as to configuration and as to details of the parts, and that the method may vary as to the specific steps and sequence, without departing from the basic concepts as disclosed herein.

Referring now to FIG. 1, a schematic view of a human eye 10 under bright lighting conditions. As shown in FIG. 1, the iris 14 is fairly constricted, leaving a relatively small pupil size (e.g. 1-3 mm). When light passes through the cornea 26, it is restricted by the iris 14 to be a fairly narrow beam 26 that hits lens 22 and passes through the vitreous humor 18, eventually striking the retina 20. If a floater 24 is present in the vitreous humor 24, a shadow 28 is cast on the on the retina. Because of the constricted iris 14, the shadow 28 makes up a substantial portion of the relatively narrow visual field 30 (the region illuminated on the retina 20).

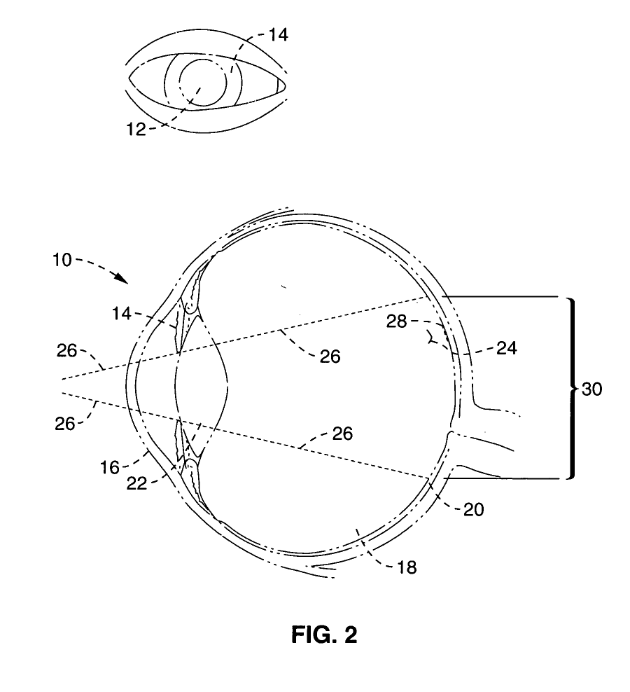

In contrast, FIG. 2 illustrates schematic view of the typical human eye under low-lighting conditions. With the expanded iris 14, the light 26 swath through the vitreous humor 18 is significantly larger, resulting in a greater illuminated region on the retina 20. In this situation, the same size floater 24 (at the same location in the vitreous) will cast a shadow 28 on the retina 20 that is proportionally (with respect to the illuminated region of the retina and the visual field 30) much smaller compared to that of lower light situations.

It has been observed that the perception of floaters is reduced, or removed altogether, when the pupil is an enlarged state, ideally between four and six millimeters in diameter. It is believed that this phenomenon is a result of the diminished or reduced presence of the floater as compared to the overall field of view. Thus, by artificially controlling the dilation of the pupil to have at least moderate dilation, the effect of floaters may be relieved and/or inhibited.

It is generally accepted that higher light levels improve visual acuity, because the smaller pupil reduces the effect of the eye's refractive errors. However, it has been shown that the relationship between light level and pupil size is important only for low light levels (Reeves, 1920). Accordingly, there is a point in which increasing light levels do not improve visual acuity due to pupil size. Thus, many patients experiencing visual anomalies may benefit from an increased pupil size that is moderately dilated, i.e. having a smaller diameter than normally mydriasis, e.g. sub-mydriatic.

For purposes of this description, mydriasis is herein defined as a condition in which the pupil is dilated to a diameter greater than approximately 6 mm combined with resistance to constriction even when stimulated with light. Mydriasis may occur in conditions such as injury of the pupillary fibers in the oculomotor nerve, in acute angle-closure glaucoma, and in ADIE syndrome, or when a dilating agent of customary concentrations has been administered in the eye. Furthermore, sub-mydriatic is defined as moderate dilation of the pupil, wherein the pupil is resistant to constriction when stimulated with light, yet having a diameter less than approximately 6 mm.

Controlled pupillary dilation may be achieved through a number of techniques. For example, pupil size may be manipulated via surgical means. Alternatively, implants or contact lenses, artificial iris/diaphragms may be installed to control the size of the pupil.

In a preferred embodiment, control of pupillary dilation is achieved through administration of a dilating agent to the surface of the eye. This has shown to be well tolerated and effective in eliminating the undesirable visual anomalies from vitreous floaters without causing undesirable side effects such as a change in focal length, headache, photopsia, or other side effects that would cause as much distress as the floaters themselves. Subjects treated with an ophthalmic solution comprising one or more of the following dilators: naphazoline, ephedrine, naphazoline (HCl or similar), phenylephrine (HCl), epinephrine and/or dipivefrin have experienced reduced incidence of vitreous floaters due to the pupil dilating effects of the aforementioned compounds.

Compositions suitable for use in the present invention include ophthalmic solutions comprising a therapeutically effective amount or dosage of one or more antihistamines, vasoconstrictors, glaucoma agents or diagnostic agents. More specifically, a therapeutically effective dosage comprises an amount sufficient to affect the iris to produce a predetermined pupil dilation, thereby causing the patient to obtain relief from vitreous floaters in their visual field.

A suitable ophthalmic solution containing an over-the-counter antihistamine may comprise naphazoline having a concentration in the range of 0.025-0.05%. An administered vasoconstrictor may have one or more of the following agents at the following concentrations: ephedrine at a range of 0.123-0.25%, naphazoline HCl at 0.01-0.05% and phenylephrine HCl at 0.05-0.1%. Administered glaucoma agents may be epinephrine at 0.5%-2%, or dipivefrin in a range of concentration from 0.05-0.1%.

The above ranges are well within the current approved levels for over-the-counter antihistamines or vasoconstrictors. In addition, the effective amounts of the glaucoma agents or diagnostic agents are well within the FDA approved levels for these compounds. In addition, all of the above described compounds are currently commercially available.

In a preferred embodiment, the one or more dilating agents are applied in carrier solution, such as an ophthalmic aqueous solution having a pH of approximately 5.6-7.0. Ideally the carrier solution would be clear and odorless. Furthermore, the carrier may be configured such that the agents are delivered in either a rapid delivery or a sustained delivery to the patient.

Because many of the above-described compounds have differing effects from patient to patient, the concentration of the compound administered may be dependent on the subject to whom the ophthalmic solution is administered. For example, a light eyed (iris) person will require less of the dilating compound to achieve pupil dilation and the subsequent relief from the vitreous floaters than a dark-eyed (iris) individual, who may require more of the dilating agent. Other factors may include age, underlying ocular disease(s) and/or conditions, and concurrent medications (drops or pills) taken by the patient.

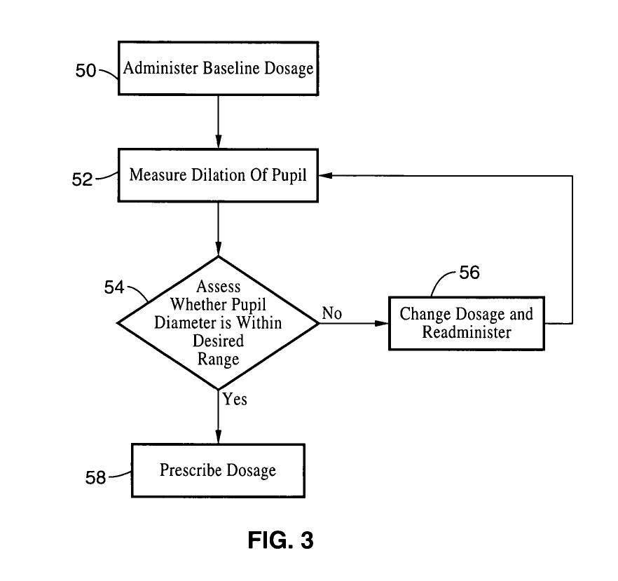

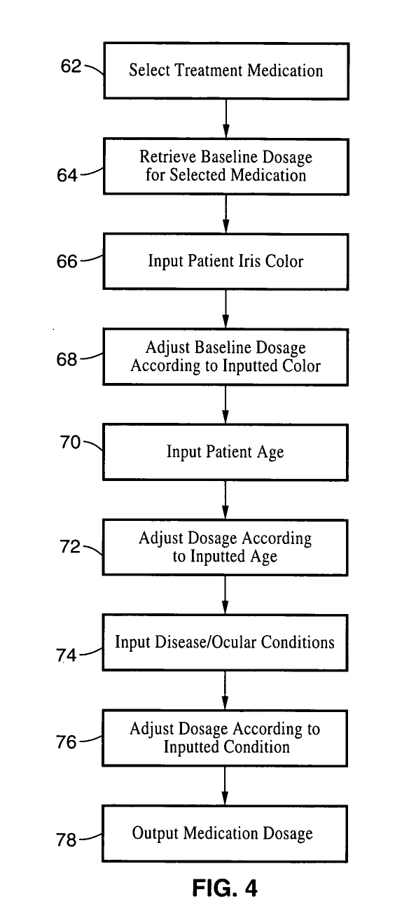

For over-the-counter medications (e.g. antihistamines or vasoconstrictors), a series of different dosages may be made available to the consumer. For example, there may be a “maximum strength” dosage for people with darker eyes, certain ailments, or low lighting conditions. Correspondingly, a “high sensitivity” dosage may also be made available for patients with lighter eyes or subject to bright lighting conditions, and various dosages may be available for conditions in between the two extremes. More precise dosages may be determined by methods administered by either the patient or the physician, such as those explained below with reference to FIGS. 3 and 4.

FIG. 3 illustrates one method using an iterative approach for determining the therapeutically effective dosage to be administered or prescribed to the patient. First, the patient is given a baseline dosage of the desired treatment medication (step 50). The baseline dosage may be the minimum amount that would be prescribed for the particular dilating agent the physician desires to use. Alternatively, the baseline dosage may be the expected dosage that the physician predicts to be effective based on one or more patient characteristics (e.g. a dosage calculated using the method shown in FIG. 4, described in detail below).

The administered medication is then given time to take effect. This may be facilitated by use of a rapid delivery carrier. At step 52, the diameter of the pupil is measured, and assessed against the desired pupil range for alleviation floaters. As explained above, the desired treatment diameter may range from under 4 mm to above 6 mm. However, more defined ranges may be desired for particular patients and for use under different conditions. For example, some patients may be particularly sensitive to the increased dilation, and thus may have a target diameter at or around 4 mm. Alternatively, other patients may have a condition that requires maximum dilation, and therefore have a target dilation closer to 6 mm. In addition, the same patient may desire different a different target dilation according to the ambient lighting, with more bright conditions demanding a lower target diameter.

If the measured diameter is not within the desired range, the dosage is adjusted and re-administered at step 56. If the physician has chosen the approach of applying a minimum baseline dosage at step 50, then another drop is immediately administered to increase the accumulative amount delivered (generally, the minimum baseline dosage is an amount configured not to exceed the desired range for any type of patient). The pupil diameter is then again measured at step 52 and assessed against the desired range at 54. If the range is not achieved, then another dose is delivered to increase the cumulative amount, and the process is repeated until the desired range is achieved. At this point, the accumulative dosage is recorded and prescribed to the patient and step 58.

Where the physician has alternatively administered a predicted baseline dosage that is expected to result in the desired dilation range, and instead results in a diameter lower than the baseline range, then the process of immediate delivery of accumulative and iterative doses, as explained above is applied. If, however, the measured diameter is over the desired range (e.g. the physician predicted too high a dosage), then the re-administration of the dosage (step 56) is done a later time after the medication has been given sufficient time to wear off. At that time, the dosage may be re-administered at the full, lower dosage and again measured. If the measured diameter is still not within range, the process is repeated according whether the diameter falls above or below the desired range.

FIG. 4 illustrates another method using an alternative approach for determining the therapeutically effective dosage to be administered or prescribed to the patient. The method 60 calculates dosage based on one or more factors that generally affect the patient's response to dilating agents. The steps of method 60 may be performed by hand using charts and tables, or via integration into a software program or the like.

At step 64, a baseline dosage is retrieved based on the selected treatment medication (step 62). One or more patient inputs (e.g. iris color (step 66), patient age (step 70), existing diseases or ocular conditions, or concurrent medications taken by the patient) are then input based patient evaluation data. Each input adjusts the baseline dosage according to the inputted information (steps 68, 72, 76). For example, a patient having light blue eyes may have a multiplication factor of 1.0, and a patient with brown eyes may be assigned a factor of X, where X is greater than 1.0 to accommodate increase resistance generally associated with darker iris pigment. Similar factors may be applied to the other inputted data. Based on these factors, a recommended medication dosage is output. This output may also be used in conjunction with the method illustrated in FIG. 3 as the baseline dosage.

In general, the total dosage range of one or more of the described dilating agents should be as such as to achieve a sufficient period of time to inhibit the visual anomalies associated with vitreous floaters for a desired duration. The composition may be administered by a physician or by the individual in need of relief from the presence of vitreous floaters. The approximately one drop dosage may thereafter be topically applied on an as needed basis.

It is also appreciated that the above methods and compositions may be used to treat a number of different ocular conditions that may benefit from increased pupillary dilation. For example for those patients having low vision may benefit from controlled pupillary dilation, particularly when the patient has abnormally small pupil dilation.

It is also appreciated that the compounds and methods of the present invention may be used to as a diagnostic tool. For example, certain diagnostic tests may benefit from an intermediate level of dilation or mydriasis. Valuable data may be attained by testing the patient at different dilation increments (e.g. at diameters ranging from 1 mm to 6 mm or higher). These increments may be achieved by starting at a high-brightness condition, and iteratively upping dilating agent dosage as described above with reference to FIG. 3.

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

What is claimed is:

1. A method of temporarily treating a visual anomaly of a patient's eye, comprising:
delivering a dilating agent to the patient's eye; and
controlling dilation of the patient's pupil to have a diameter within in a predetermined range;
wherein said controlled pupil dilation results in at least a temporary reduction in the presence of said visual anomaly.

2. A method as recited in claim 1, wherein the visual anomaly comprises vitreous floaters.

3. A method as recited in claim 1, wherein the visual anomaly comprises low vision.

4. A method as recited in claim 1, wherein controlling dilation of the patient's pupil comprises controlling the diameter of the pupil to be moderately dilated.

5. A method as recited in claim 4, wherein controlling dilation of the patient's pupil comprises controlling the diameter of the pupil to be sub-mydriatic.

6. A method as recited in claim 5, wherein the pupil is controlled to have a diameter in the range of equal to or less than 7 mm.

7. A method as recited in claim 6, wherein the pupil is controlled to have a diameter in the range of about 3 mm-6.5 mm.

8. A method as recited in claim 7, wherein the pupil is controlled to have a diameter in the range of about 4 mm-6. mm

9. A method as recited in claim 1, wherein the dilating agent comprises one or more of the following: antihistamine, vasoconstrictor, glaucoma agent, or diagnostic agent.

10. A method as recited in claim 1, wherein the dilating agent comprises one or more of the following: naphazoline, ephedrine, phenylephrine, epinephrine, or dipivefrin.

11. A method as recited in claim 1, wherein the dilating agent is delivered topically.

12. A method as recited in claim 11, wherein the dilating agent is delivered in an ophthalmic carrier solution.

13. A method as recited in claim 12, wherein the carrier solution is configured such that the dilating agent is delivered in a one-drop dose.

14. A method as recited in claim 1:
wherein the dilating agent is delivered in a therapeutically effective dosage; and
wherein said therapeutically effective dosage comprises a minimum amount of dilating agent necessary to effect dilation of the patient's pupil to be within the predetermined range.

15. A method as recited in claim 11, wherein the therapeutically effective dosage is varies according to the patient's iris color.

16. A method of controlling dilation of a pupil of an eye, comprising:
topically delivering a dilating agent to surface of the eye; and
controlling dilation of the pupil;
wherein the dilating agent is delivered at a concentration that controls dilation of the pupil to a pre-determined diameter corresponding to a sub-mydriatic level.

17. A method as recited in claim 16, wherein the pre-determined diameter is less than approximately 7 mm.

18. A method as recited in claim 17, wherein the pre-determined diameter is less than approximately 6 mm

19. A method as recited in claim 18, wherein the pre-determined diameter is greater than approximately 4 mm.

20. A method as recited in claim 16, wherein the dilating agent is delivered at a concentration that results in a pre-determined pupil diameter that inhibits the effect of vitreous floaters.

21. A method as recited in claim 16, wherein the dilating agent is delivered at a concentration that results in a pre-determined pupil diameter suitable for diagnostic testing of the eye.

22. A method as recited in claim 16, wherein the dilating agent concentration is varied to accommodate different sensitivities associated with the patient's iris color.

23. A method as recited in claim 16, wherein the dilating agent concentration is varied to accommodate different sensitivities associated with the patient's age.

24. A method as recited in claim 16, wherein the dilating agent concentration is varied to accommodate different sensitivities associated with concurrent medications taken by the patient.

25. An ophthalmic solution, comprising:
an aqueous ophthalmic carrier solution configured to be topically delivered to a surface on a patient's eye; and
a dilating agent configured to control dilation of a pupil of the patient;
wherein the dilating agent has a concentration that is configured dilate the pupil to a diameter in a pre-determined, sub-mydriatic range.

26. An ophthalmic solution as recited in claim 25, wherein the dilating agent comprises one or more of the following: antihistamine, vasoconstrictor, glaucoma agent, or diagnostic agent.

27. An ophthalmic solution as recited in claim 25, wherein the carrier solution is configured to deliver the dilating agent in a one-drop dose.

28. An ophthalmic solution as recited in claim 25, wherein the dilating agent has a concentration that is configured dilate the pupil to a diameter that inhibits the effect of vitreous floaters.

29. An ophthalmic solution as recited in claim 25, wherein the dilating agent has a concentration that is configured dilate the pupil at different incremental diameters for diagnostic testing of the eye.

30. A method of prescribing a medication for treating visual anomalies of a patient's eye; comprising:
administering a baseline dosage of a dilating agent topically to the eye;
measuring the dilation of a the patient's pupil;
comparing the measured dilation against a desired dilation range; and
changing the dosage until the measured dilation is within the desired range;
wherein the desired dilation range is configured to reduce the effect of said visual anomalies.

31. A method as recite in claim 30, wherein the baseline dosage is a function of one or more of the following patient conditions: patient iris color, patient age, patient ocular conditions, and concurrent patient medications.

32. A method as recited in claim 31, wherein the desired dilation range corresponds to a range of diameters that reduces the perception of vitreous floaters.
